(19) United States
(12) Patent Application Publication
(10) Pub. No.: US 2016/0157724 A1
(43) Pub. Date: Jun. 9, 2016

(54) REGISTERING OF PHYSIOLOGICAL PARAMETERS BASED ON IMAGE ANALYSIS OF LIGHT REFLECTION

(71) Applicant: DELAVAL HOLDING AB, Tumba (SE)

(72) Inventor: Haim KRIEF, Hadera (IL)

(21) Appl. No.: 14/409,007

(22) PCT Filed: Jul. 9, 2013
(86) PCT No.: PCT/SE2013/050880

§ 371 Date: Dec. 18, 2014

Related U.S. Application Data
(60) Provisional application No. 61/669,736, filed on Jul. 10, 2012.

(30) Foreign Application Priority Data
Jul. 10, 2012 (SE) 1250807-3

(51) Int. Cl. A61B 5/00; A61B 5/11; A61B 5/0205

(52) U.S. Cl. CPC A61B 5/0077; A61B 5/6822; A61B 5/6831; A61B 5/0205; A61B 5/1118; A61B 5/7278; A61B 2503/40

(57) ABSTRACT

An apparatus includes a housing with a flexible wall having an outer side configured to contact an external body surface of an animal and an inner side with a light reflective surface towards an interior of the housing. A light source illuminates the light reflective surface, and an image registering part captures image data representing the light reflective surface. A data processing part receives the image data, and based thereon produces at least one signal indicative of at least one physiological parameter of the animal. The apparatus may be arranged on the animal such that the outer side of the flexible wall contacts a neck region of the animal and the data processing part derives a primary signal representing a chewing related parameter.

### THE BACKGROUND OF THE INVENTION AND PRIOR ART

The present invention relates generally to solutions for determining physiological parameters of an animal. More particularly the invention relates to an apparatus according to the preamble of claim 1, a neckband according to claim 6 and a method according to the preamble of claim 8. The invention also relates to a computer program according to claim 13 and a computer readable medium according to claim 14.

To accomplish an efficient and animal friendly livestock handling it is important that the animals' physiological state and health condition be monitored. Of course, to this aim, regular farmer's inspections and veterinary examinations can never be excluded. However, as a complement thereto and to provide an ongoing supervision, various automatic systems can be employed. Today, a so-called activity meter may be employed, which is arranged in a neckband on the animal in order to register and report the animal's activity level to a remote location, e.g. via radio. The activity level is indicative of whether or not the animal is healthy. Nevertheless, the activity level is a very crude measure. Therefore, for ruminating animals such as cows, chewing behavior, rumen activity, cardiac activity and respiration would provide more valuable data. Known sensors for measuring cardiac activity are often based on the well known technology of pulse oximetry, as described e.g. in US 2009/0105605.

US 2010/0226543 discloses solutions for imaging objects, for example living body parts, wherein the objects are illuminated from a distance and surface vibrations thereof are image analyzed to extract various data, e.g. representing speech or heart beats.

### PROBLEMS ASSOCIATED WITH THE PRIOR ART

It is known to use pulse oximetry for measuring the heart rate of animals. Further, general solutions are known wherein, based on advanced image analysis, signals are derived that originate from internal body vibrations (or sounds). However, there is no example of a practical and cost-efficient solution for using this type of technology instead of e.g. pulse oximetry for registering physiological signals of animals in the field, e.g. in a farm environment.

### SUMMARY OF THE INVENTION

The object of the present invention is to solve the above problem, and thus offer a reliable, robust and animal-friendly solution for registering signals indicative of physiological parameters of a given animal.

According to one aspect of the invention, the object is achieved by the initially described apparatus, wherein the apparatus includes a housing. The housing, in turn, contains a flexible wall, a light source, an image registering means and a data processing means. The flexible wall has an outer side configured to contact an external body surface of an animal. An inner side of the flexible wall contains a light reflective surface towards an interior of the housing. The light source is configured to illuminate the light reflective surface, and the image registering means is configured to capture image data representing the light reflective surface. The data processing means is configured to receive the image data, and based thereon; produce at least one signal indicative of at least one physiological parameter of the animal.

This apparatus is advantageous because it enables registering of highly complex signals in a very uncomplicated and straight-forward manner.

According to one preferred embodiment of this aspect of the invention, the apparatus is configured to be arranged on the animal, such that the outer side of the flexible wall contacts a neck region of the animal. The data processing means is specifically configured to derive a primary signal representing a chewing related parameter. Thereby, important information concerning the animal's physiological status is obtainable, for instance related to food intake and digestion. Further preferably, the data processing means is configured to derive at least one secondary signal representing: rumen activity, a cardiogram, heart rate, respiration rate, and/or a general activity level of the animal.

According to another preferred embodiment of this aspect of the invention, the light source includes a green laser. Green laser light is associated with a relatively low penetration, and therefore a large proportion of such light will be reflected back from the light reflective surface of the flexible wall, which is equivalent to a high degree of efficiency in terms of energy used.

According to a yet another preferred embodiment of this aspect of the invention, the data processing means is configured to determine a strongest light reflection in each image registered by an image sensor in the image registering means. From a set of such strongest light reflections determined in a series of images, the data processing means is further configured to derive the at least one signal. Hence, an uncomplicated time-varying signal is produced, which may serve as a basis for further analysis.

According to another aspect of the invention, the object is achieved by a neckband adapted to be carried around the neck of a cow. The neckband includes the above-proposed apparatus and a fitting member configured to grip around the back of the neck of the cow, so as to reduce rotation movements of the neckband relative to the cow's neck. Thus, the apparatus is held in a relatively fix position without requiring a high pressure against the animal, which is advantageous from an animal-comfort point-of-view.

According to one preferred embodiment of this aspect of the invention, the neckband includes a weight member arranged essentially opposite to the fitting member on the neckband. The weight member is configured to pull the fitting member towards the back of the neck of the cow, and the proposed apparatus is arranged between the fitting member and the weight member. As a result, when the neckband is carried around the neck of a cow, the flexible wall will contact an external body surface of the animal while the risk of rotation movements of the neckband relative to the cow's neck is held relatively low and the flexible wall exerts a relatively low pressure on the external body surface. Of course, the weight member, in turn, may contain useful components, such a transponder and/or units for radio communication.

According to another aspect of the invention, the object is achieved by the method described initially, which presumes the use of an apparatus that has a housing including a flexible wall with an outer side and an inner side that is light reflective. The method involves arranging the apparatus on an animal, such that the flexible wall contacts an external body surface of the animal. The light reflective surface is illuminated from an interior of the housing, and image data are captured representing the reflective surface. The image data are processed to produce at least one signal indicative of at least one physiological parameter of the animal. The advantages of this method, as well as the preferred embodiments thereof, are apparent from the discussion above with reference to the proposed apparatus.

According to a further aspect of the invention the object is achieved by a computer program, which is directly loadable into the memory of a computer, and includes software adapted to implement the method proposed above when said program is run on a computer.

According to another aspect of the invention the object is achieved by a computer readable medium, having a program recorded thereon, where the program is to control a computer to perform the method proposed above when the program is loaded into the computer.

Further advantages, beneficial features and applications of the present invention will be apparent from the following description and the dependent claims.

### BRIEF DESCRIPTION OF THE DRAWINGS

The invention is now to be explained more closely by means of preferred embodiments, which are disclosed as examples, and with reference to the attached drawings.

FIG. 1 shows a ruminant animal carrying a neckband containing the proposed apparatus;

FIG. 2 illustrates an apparatus according to one embodiment of the invention that is arranged on an animal;

FIG. 3 shows an image sensor according to one embodiment of the invention illustrating an example of a set of strongest light reflections registered in a set of images;

FIG. 4 shows an example of a time-varying function derived from the set of strongest light reflections in a set of images in FIG. 3; and

FIG. 5 illustrates, by means of a flow diagram, the general method according to the invention.

### DESCRIPTION OF PREFERRED EMBODIMENTS OF THE INVENTION

We refer initially to FIG. 1, which shows a ruminant animal A carrying a neckband 100 containing the proposed apparatus 110 around its neck. The neckband 100 has a fitting member 130 configured to grip around the back of the cow's neck, so as to reduce rotation movements of the neckband 100 relative to the cow's neck. The fitting member 130 may thus include a section (e.g. of plastic) that is pre-shaped to fit the general contour of the back of the cow's neck.

According to one preferred embodiment of the invention, the neckband 100 includes a weight member 120, which is arranged essentially opposite to the fitting member 130 on the neckband 100, and is configured to pull the fitting member 130 towards the back of the neck of the cow. The apparatus 110 is arranged between the fitting member 130 and the weight member 120, such that when the neckband 100 is carried around the neck of a cow a sensor part of the apparatus 110 is drawn towards and to contact an external body surface of the animal A so that the sensor part of the apparatus 110 contacts the external body surface. At the same time, the risk that the neckband 100 rotates relative to the cow's neck is held relatively low, and said sensor part exerts a relatively low pressure on the animal's A body. The weight member 120 preferably contains high-density components, such as batteries and/or radio components, e.g. for transmitting any signals S(t) produced by the apparatus 110.

Naturally, according to the invention, in addition to the proposed apparatus 110 and any weight member 120, the neckband 100 may include one or more other types of sensors, preferably configured to register signals different from those registered by the apparatus 110.

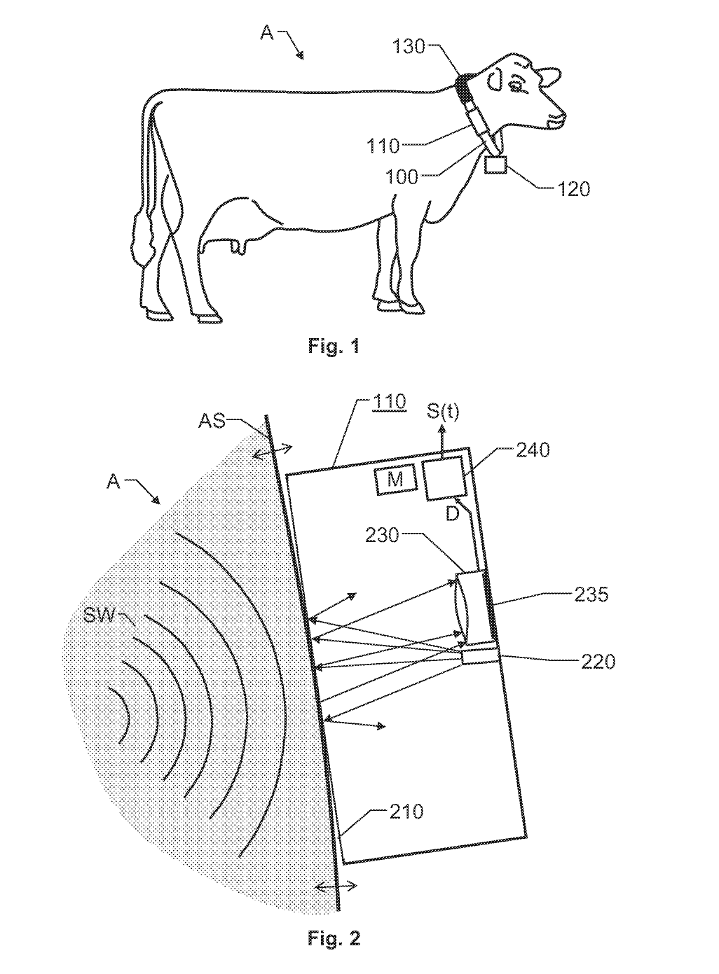

Referring to FIG. 2, more specifically, a flexible wall 210 of the apparatus 110 will exert a relatively low pressure on an external body surface AS of the animal A while the risk of rotation movements of the neckband 100 relative to the cow's neck is held relatively low. FIG. 2 shows a side view of the apparatus 110 according to one embodiment of the invention when arranged on an animal A.

The apparatus 110 includes a housing, which, in turn, contains: a flexible wall 210, a light source 220, an image registering means 230 and a data processing means 240.

The flexible wall 210 represents the above-mentioned sensor part and has an outer side configured to contact the external body surface AS of the animal A. An inner side of the flexible wall 210 contains a light reflective surface towards an interior of the housing.

The light source 220 is configured to illuminate the light reflective surface. Preferably, the light source 220 contains a green laser because due to the relatively low degree of penetration of green laser light, such a light source is associated with comparatively high degree of energy efficiency.

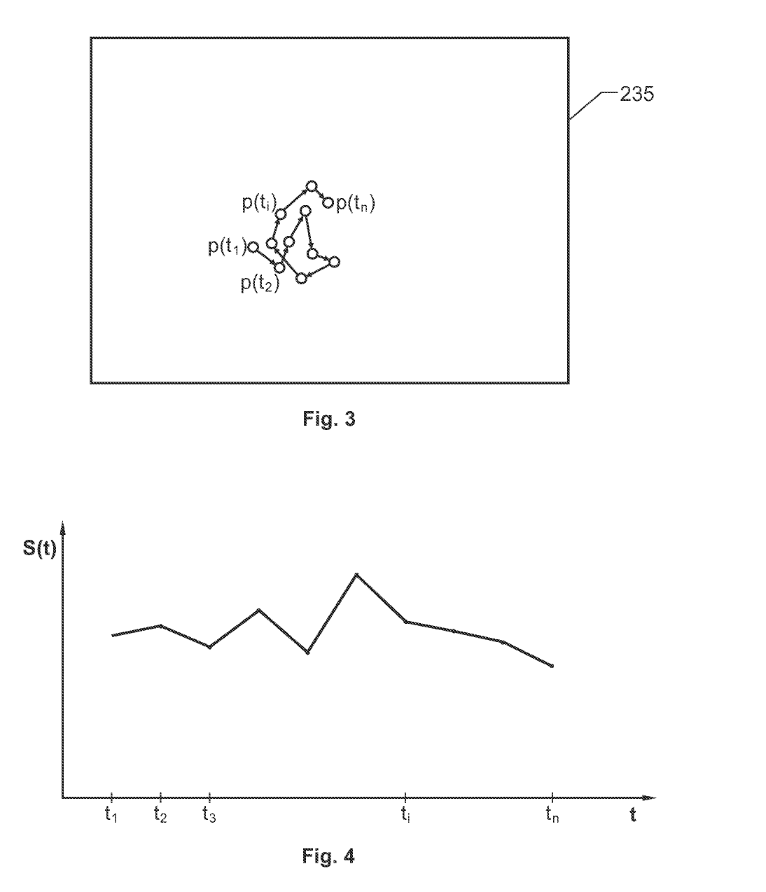

The image registering means 230 is configured to capture image data D representing the light reflective surface. To this aim, the image registering means 230 includes an image sensor 235 (see FIG. 3).

The data processing means 240 is configured to receive the image data D, and based thereon; produce at least one signal S(t) indicative of at least one physiological parameter of the animal A.

As mentioned above, the apparatus 110 is preferably configured to be arranged on the animal A, such that the outer side of the flexible wall 210 contacts a neck region of the animal A. Thereby, any signals SW (typically sounds, i.e. mechanical waves) originating from internal organs in the animal A may propagate through the animal's A body and cause vibrations of the external body surface AS of the animal A. Consequently, by studying the vibrations of the external body surface AS, it is possible to draw conclusions concerning the animal's A chewing behavior, rumen activity, cardiac activity (cardiogram and/or heart rate) as well as respiration rate. In fact, a general activity level of the animal A may also be derived, since animal activity corresponds to low-frequency noise of high magnitude (relative to the other signals).

According to embodiments of the invention, the data processing means 240 is configured to derive a primary signal S(t) representing a chewing related parameter. Additionally, the data processing means 240 may also be configured to derive at least one secondary signal S(t) representing: rumen activity, a cardiogram, heart rate, respiration rate and/or a general activity level of the animal A.

FIG. 3 shows the image sensor 235 according to one embodiment of the invention. Here, we see an example of a set of strongest light reflections p(t₁), p(t₂), . . . p(tᵢ), . . . p(tₙ) registered in a set of images, say n images. Thus, each image essentially results in a point in a two-dimensional plane, where the point represents the location of the strongest light reflection in that image. This, in turn, may be translated by the data processing means 240 into a one-dimensional function S(t) of time t as illustrated in FIG. 4.

In FIG. 4 we an example of a time-varying function S(t) derived from the set of strongest light reflections p(t₁), p(t₂), . . . p(tᵢ), . . . p(tₙ) in FIG. 3, where the magnitude of S(t) is equivalent to a distance between two consecutive strongest light p(tᵢ). Preferably, the data processing means 240 is configured to apply a threshold, such that a strongest light reflection corresponding to an exceptionally large magnitude is discarded. Naturally, according to the invention, many alternative (and more complex) parameters may also be derived from the image data D captured by the image sensor 235.

The above procedure implemented by the processing means 240 is preferably controlled by a computer program M loaded into a memory of the processing means 240, or an external memory unit accessible by the processing means 240. The computer program, in turn, contains software for controlling the steps of the procedure when the program is run on the processing means 240.

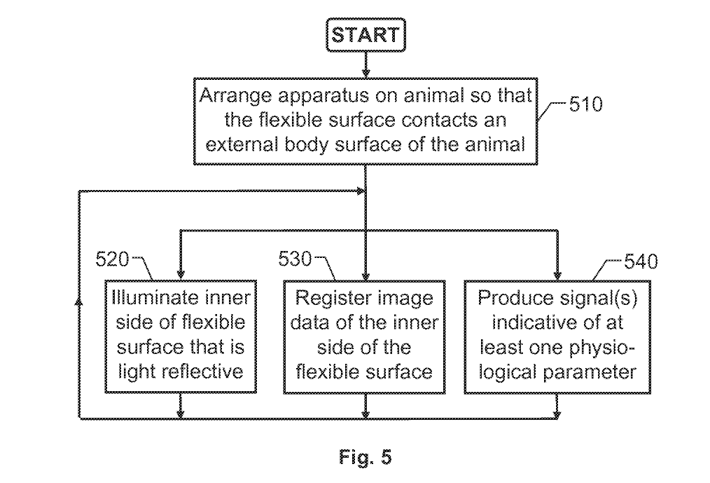

In order to sum up, we will now describe the general method according to the invention with reference to the flow diagram in FIG. 5.

In a first step 510, an apparatus 110 is arranged on an animal A, so that an outer side of its flexible wall 210 contacts an external body surface AS, preferably in the neck region.

In a following step 520, the light reflective surface is illuminated from an interior of the apparatus' 110 housing. A step 530, parallel to step 520, registers image data D representing the reflective surface of the inner side of the flexible wall 210. A step 540, parallel to steps 520 and 530, processes the image data D to produce at least one signal S(t) indicative of at least one physiological parameter of the animal A. Then, the procedure loops back to steps 520, 530 and 540 for repeated measurement.

All of the process steps, as well as any sub-sequence of steps, described with reference to FIG. 5 above may be controlled by means of a programmed computer apparatus. Moreover, although the embodiments of the invention described above with reference to the drawings comprise computer apparatus and processes performed in computer apparatus, the invention thus also extends to computer programs, particularly computer programs on or in a carrier, adapted for putting the invention into practice. The program may be in the form of source code, object code, a code intermediate source and object code such as in partially compiled form, or in any other form suitable for use in the implementation of the process according to the invention. The program may either be a part of an operating system, or be a separate application. The carrier may be any entity or device capable of carrying the program. For example, the carrier may comprise a storage medium, such as a Flash memory, a ROM (Read Only Memory), for example a DVD (Digital Video/Versatile Disk), a CD (Compact Disc) or a semiconductor ROM, an EPROM (Erasable Programmable Read-Only Memory), an EEPROM (Electrically Erasable Programmable Read-Only Memory), or a magnetic recording medium, for example a floppy disc or hard disc. Further, the carrier may be a transmissible carrier such as an electrical or optical signal which may be conveyed via electrical or optical cable or by radio or by other means. When the program is embodied in a signal which may be conveyed directly by a cable or other device or means, the carrier may be constituted by such cable or device or means. Alternatively, the carrier may be an integrated circuit in which the program is embedded, the integrated circuit being adapted for performing, or for use in the performance of, the relevant processes.

Although the invention has been described primarily with reference to cows, the invention is equally well adapted for any other kind of animals, such as goats, sheep or buffaloes.

The term “comprises/comprising” when used in this specification is taken to specify the presence of stated features, integers, steps or components. However, the term does not preclude the presence or addition of one or more additional features, integers, steps or components or groups thereof.

The invention is not restricted to the described embodiments in the figures, but may be varied freely within the scope of the claims.

1-14. (canceled)

15. An apparatus (110) that registers signals indicative of physiological parameters of an animal (A) based on illumination of a surface and analysis of light reflections from the surface, said apparatus comprising:
a housing with a flexible wall (210) having an outer side and an inner side, the inner side of the flexible wall containing a light reflective surface oriented towards an interior of the housing, wherein the outer side of the flexible wall is configured to contact an external body surface (AS) of the animal (A) and exert a pressure sufficient that mechanical waves, originating from internal organs in the animal and causing vibrations of the external body surface of the animal, cause flexing of the light reflective surface;
a light source (220) that, in use, illuminates the light reflective surface such that light from the light source is reflected off the light reflective surface, the light reflected off the light reflective surface representing the vibrations of the external body surface of the animal;
an image registering part (230) with a sensor (235) that, in use, captures the light reflected off the light reflective surface and extracts image data (D) from the captured light, the image data (D) representing the vibrations of the external body surface of the animal; and
a data processing part (240) connected to receive the image data (D) from the image registering part, and, based on processing the received image data (D), produces a time-varying signal (S(t)) based on a strongest light reflected off the light reflective surface at each of a series of points in time, the time-varying signal (S(t)) being indicative of at least one physiological parameter of the animal (A).

16. The apparatus (110) according to claim 15, wherein,
the time-varying signal (S(t)) indicative of the at least one physiological parameter of the animal (A) represents a one-dimensional time-varying function indicative of the at least one physiological parameter of the animal (A),
the sensor is an image sensor that produces a set of images of the light reflected off the light reflective surface at each of the points in time, and
the data processing part (240) uses the set of images to detect a set of strongest light reflections (p(t1) . . . p(tn)) registered in a set of images at each of the points in time, the strongest light reflect of each image resulting in a point in a two-dimensional plane that represents a location of the strongest light reflection in that image, and the data processing part (240) translates the location of the strongest light reflection in each image of the image set into the one-dimensional time-varying function.

17. The apparatus (110) according to claim 15, wherein,
the sensor is an image sensor that produces a set of images of the light reflected off the light reflective surface at each of the points in time, and
the data processing part (240) uses the set of images to detect a set of strongest light reflections (p(t1) . . . p(tn)) registered in a set of images at each of the points in time, the strongest light reflect of each image resulting in a point in a two-dimensional plane that represents a location of the strongest light reflection in that image, and
the location of the strongest light reflection in each image of the image set translates into a time-varying function S(t).

18. The apparatus (110) according to claim 15, wherein the time-varying signal (S(t)) derived by the data processing part (240) represents a chewing parameter of the animal.

19. The apparatus (110) according to claim 18, wherein the data processing part (240) is configured to derive at least one secondary time-varying signal (S(t)) representing at least one of the group consisting of: rumen activity, a cardiogram, heart rate, respiration rate, and a general activity level of the animal (A).

20. The apparatus (110) according to claim 15, wherein the light source (220) comprises a green laser.

21. The apparatus (110) according to claim 15, further comprising:
a neckband (100) adapted to be carried around the neck of a cow, and
a fitting member (130) configured to grip around a back of the neck of the cow, the fitting member reducing rotation movements of the neckband (100) relative to the cow's neck.

22. The apparatus (110) according to claim 21, further comprising a weight member (120) arranged essentially opposite to the fitting member (130) on the neckband (100),
wherein the weight member pulls the fitting member (130) towards the back of the neck of the cow, and
wherein the housing is arranged between the fitting member (130) and the weight member (120) such that when the neckband is carried around the neck of a cow, the flexible wall (210) contacts the external body surface (AS) of the cow and a risk of rotation movements of the neckband (100) relative to the cow's neck is held relatively low and the flexible wall (210) exerts a relatively low pressure on the external body surface (AS).

23. A method for registering signals indicative of physiological parameters of an animal (A) based on illumination of a surface and analysis of light reflections from the surface, said method comprising:
placing an apparatus (110) comprising a housing, with the flexible wall (210) having an outer side and an inner side, around a neck of the animal such that the outer side of the flexible wall (210) contacts an external body surface (AS) of the animal (A), wherein the inner side of the flexible wall contains a light reflective surface oriented towards an interior of the housing, the outer side of the flexible wall is placed in contact the external body surface (AS) of the animal (A) so as to exert a pressure sufficient that mechanical waves, originating from internal organs in the animal and causing vibrations of the external body surface of the animal, cause flexing of the light reflective surface, and the apparatus further comprises i) a light source (220) that illuminates the light reflective surface such that light from the light source is reflected off the light reflective surface, the light reflected off the light reflective surface representing the vibrations of the external body surface of the animal, ii) an image registering part (230) with a sensor (235) that captures the light reflected off the light reflective surface and extracts image data (D) from the captured light, the image data (D) representing the vibrations of the external body surface of the animal, and iii) a data processing part (240) that receives the image data (D) from the image registering part, and, based on the received image data (D), produces a time-varying signal (S(t)) based on a strongest light reflected off the light reflective surface at each of a series of points in time, the time-varying signal (S(t)) being indicative of at least one physiological parameter of the animal (A);
with the light source, illuminating the light reflective surface from the interior of the housing;
with the image registering part, capturing the light reflected off the light reflective surface and extracting image data (D) from the captured light, the image data (D) representing the vibrations of the external body surface of the animal; and
with the data processing part, processing the image data (D) to produce a time-varying signal (S(t)) based on the strongest light reflected off the light reflective surface at each of a series of points in time, the time-varying signal (S(t)) being indicative of at least one physiological parameter of the animal (A).

24. The method according to claim 23, wherein,
the animal is a cow,
the time-varying signal (S(t)) indicative of the at least one physiological parameter of the animal (A) represents a one-dimensional time-varying function indicative of the at least one physiological parameter of the cow,
the capturing step produces a set of images of the light reflected off the light reflective surface at each of the points in time, and
in the processing step, the data processing part (240) uses the set of images to detect a set of strongest light reflections (p(t1) . . . p(tn)) in a set of images at each of the points in time, the strongest light reflect of each image resulting in a point in a two-dimensional plane that represents a location of the strongest light reflection in that image, and the data processing part (240) translates the location of the strongest light reflection in each image of the image set into the one-dimensional time-varying function.

25. The method according to claim 23, wherein,
the capturing step produces a set of images of the light reflected off the light reflective surface at each of the points in time, and
in the processing step, the data processing part (240) uses the set of images to detect a set of strongest light reflections (p(t1) . . . p(tn)) in a set of images at each of the points in time, the strongest light reflect of each image resulting in a point in a two-dimensional plane that represents a location of the strongest light reflection in that image, the location of the strongest light reflection in each image of the image set translating into a time-varying function S(t).

26. The method according to claim 23, wherein, in the processing step, the data processing part (240) uses the time-varying signal (S(t)) to derive a time-varying function representing a chewing parameter of the animal.

27. The method according to claim 23, wherein,
the animal is a cow, and
in the processing step, the data processing part (240) derives at least one secondary time-varying function representing at least one of the group consisting of: rumen activity, a cardiogram, heart rate, respiration rate, and a general activity level of the cow.

28. The method according to claim 23, wherein in the illuminating step, the light source uses a green laser (220) to illuminate the light reflective surface from the interior of the housing.

29. The method according to claim 23, wherein the processing step comprises:
determining a strongest light reflection ((p(t1), . . . , (p(tn)) in each image registered by an image sensor (235) in the image registering part (230), and
deriving the at least one signal (S(t)) from a set of strongest light reflections ((p(t1), . . . , (p(tn)) determined in a series of images.

30. A non-transitory computer readable medium (M) having a program recorded thereon, the program when executed on a computer controls an apparatus (110) to register signals indicative of physiological parameters of an animal (A), wherein said apparatus comprises:
a housing with a flexible wall (210) having an outer side and an inner side, the inner side of the flexible wall containing a light reflective surface oriented towards an interior of the housing, wherein the outer side of the flexible wall is configured to contact an external body surface (AS) of the animal (A) and exert a pressure sufficient that mechanical waves, originating from internal organs in the animal and causing vibrations of the external body surface of the animal, cause flexing of the light reflective surface;
a light source (220) that illuminates the light reflective surface such that light from the light source is reflected off the light reflective surface, the light reflected off the light reflective surface representing the vibrations of the external body surface of the animal;
an image registering part (230) with a sensor (235) that captures the light reflected off the light reflective surface and extracts image data (D) from the captured light, the image data (D) representing the vibrations of the external body surface of the animal; and
a data processing part (240) connected to receive the image data (D) from the image registering part, and, based on processing the received image data (D), produces a time-varying signal (S(t)) based on a strongest light reflected off the light reflective surface at each of a series of points in time, the time-varying signal (S(t)) being indicative of at least one physiological parameter of the animal (A).

31. The non-transitory computer readable medium (M) according to claim 30, wherein,
the time-varying signal (S(t)) indicative of the at least one physiological parameter of the animal (A) represents a one-dimensional time-varying function indicative of the at least one physiological parameter of the animal (A),
the sensor is an image sensor that produces a set of images of the light reflected off the light reflective surface at each of the points in time, and
the data processing part (240) uses the set of images to detect a set of strongest light reflections (p(t1) . . . p(tn)) registered in a set of images at each of the points in time, the strongest light reflect of each image resulting in a point in a two-dimensional plane that represents a location of the strongest light reflection in that image, and the data processing part (240) translates the location of the strongest light reflection in each image of the image set into the one-dimensional time-varying function.

32. The non-transitory computer readable medium (M) according to claim 30, wherein,
the sensor produces a set of images of the light reflected off the light reflective surface at each of the points in time, and
the data processing part (240) uses the set of images to detect a set of strongest light reflections (p(t1) . . . p(tn)) registered in a set of images at each of the points in time, the strongest light reflect of each image resulting in a point in a two-dimensional plane that represents a location of the strongest light reflection in that image, and
the location of the strongest light reflection in each image of the image set translates into a time-varying function S(t).

33. The non-transitory computer readable medium (M) according to claim 30, wherein the time-varying signal (S(t)) derived by the data processing part (240) represents a chewing parameter of the animal.

34. The non-transitory computer readable medium (M) according to claim 30, wherein the data processing part (240) derives at least one secondary time-varying signal (S(t)) representing at least one of the group consisting of:
rumen activity, a cardiogram, heart rate, respiration rate, and a general activity level of the animal (A).

35. The method according to claim 23, wherein the data processing part (240) derives at least one secondary time-varying signal (S(t)) representing rumen activity of the animal (A).
